(19) United States
(12) Patent Application Publication
(10) Pub. No.: US 2008/0054223 A1
(43) Pub. Date: Mar. 6, 2008

(54) Light Emitting Complex Salts

(75) Inventors: Martyn John Earle, Belfast (GB); Kenneth Richard Seddon, County Down (GB)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: The Queen's University of Belfast, Belfast (IE)

(21) Appl. No.: 11/665,960

(22) PCT Filed: Oct. 24, 2005
(86) PCT No.: PCT/GB05/04121

§ 371 Date: Sep. 20, 2007

(30) Foreign Application Priority Data
Oct. 22, 2004 (GB) 0423506.5

(51) Int. Cl. C09K 11/06; C07D 213/04; C07D 233/54; H01L 51/30; C07D 403/04; C07F 7/00

(52) U.S. Cl. 252/301.16; 534/15; 540/541; 544/225; 546/9; 548/107

(57) ABSTRACT

Complex salts, based on ionic liquids, which exhibit at least one light emitting property selected from (a) fluorescence, (b) phosphorescence, and (c) electroluminescence when in the solid state; and which have a melting point below 250° C.

This invention relates to light-emitting complex salts, and uses thereof.

Compounds having various light-emitting characteristics (e.g. fluorescence, phosphorescence, electroluminescence, etc.) find utility in a wide range of industrial applications. Examples include imaging and display devices, electro-optical devices and assay procedures. For example, fluorescent, phosphorescent and electroluminescent compounds find wide application in the manufacture of cathode ray tubes, fluorescent tubes, X-ray-imaging screens, radiation detectors, toys and other recreational devices, signs, light-emitting solid state devices etc. Generally inorganic phosphors are used in such applications and these have the disadvantage that they require complex deposition techniques.

Other display devices are passive in the sense of utilising components that modulate another light source. Examples include liquid crystal displays of the kind found in mobile telephones, calculators, computer screens and flat-screen television displays. Although more convenient to manufacture than cathode ray tube displays, such devices require a separate light source and the materials from which they are manufactured tend to deteriorate with time.

The present invention seeks to address these problems and has done so by providing a new class of light emitting compounds that comprise complex salts formed between a complexed metal anion and a selected organic cat ion. It has been found that by appropriate selection of the complexed metal anion and the organic cation, compounds having a wide range of desirable physical properties may be produced. For example, the basic light emitting properties of the complexes may be predetermined by appropriate selection of the metal and its associated ligand. Similarly, properties such as melting point and solubility in organic solvents may be determined by appropriate selection of the organic cation. It has also been found that the organic cation can affect the luminescent properties of the complex as a whole.

Relatively high melting-point triboluminescent manganese-based complexes with tertiary alkylammonium and tertiary methylphenyl phosphonium compounds have been described by Cotton, F. A. et al. and Hardy, G. E. et al. (See: “Correlation of Structure and Thboluminescence for Tetrahedral Manganese (II) Compounds”, Cotton F. A. et al., Inorg. Chem. 2001, 40, 3576-3578; “Triboluminescence and Pressure Dependence of the Photoluminescence of Tetrahedral Manganese (II) Complexes”, Gordon E. H. et al. Inorg. Chem., Vol. 15, No. 12, 1976 pp 3061).

According to one aspect of the present invention there is provided the use of complex salts having the formula
([Org]ⁿ⁺)ₘ·([M(Lg)pf)ₙ (A)
wherein m=1, 2, 3 or 4;
   n=1 or 2;
   p=3, 4, 5 or 6;
   M is a metal;
   each Lg, which may be the same or different, represents a ligand; and
   [Org]^{π+} represents an organic cation
      in the manufacture of a luminescent display device, in the manufacture of a coating material, e.g. a paint, or for incorporation into a plastics composition. By “luminescent display device” is meant a device wherein in use, the device produces a fluorescent, phosphorescent or electroluminescent light signal. The device is preferably used for visual display applications. The device is preferably used for visual display applications. Examples of coating materials include paints and inks.

Complex salts having the formula (A) and which (1) exhibit at least one light emitting property selected from (a) fluorescence, (b) phosphorescence, and (c) electroluminescence when in the solid state, (2) have a melting point below 250° C., preferably below 200° C. ₁ and (3) are capable of forming ionic liquids when molten are novel and form a further aspect of the present invention.

The invention further provides complex salts having the formula
([Org]ⁿ⁺)ₘ·([M(Lg)J ⁿ⁻)ₙ (A)
wherein m=1, 2, 3 or 4;
   n=1 or 2;
   p=3, 4, 5 or 6;
   M is a metal;
   each Lg, which may be the same or different, represents a ligand; and
   [OrgJⁿ⁺ represents an organic cation
      with the proviso that when M is Mn, the organic cation [Org]^{π+} is (a) other than tetramethylammonium, tetraethylammonium, tetrabutylammonium, trimethylphenylphosphonium and triphenylmethylphosphonium,

For a given anion, ([M(Lg)ₚ]^{m-})_{n′} complex salts according to the invention can be produced with a range of selected physical properties, such as melting point and solubility in organic solvents. Thus, complex salts according to the invention may have melting points below 180° C., below 150° C., below 125° C. and in some instances below 100° C.

The values of m, n and p will depend upon the valence state and coordination number of metal M. Typically, for a four-coordinated metal ion in the +2 oxidation state, such as manganese (II), m will be 2, n will be 1 and p will be 4. With other metal ions, p may have other values, e.g. 5 or 6.

Examples of metals “M” include Group VII or VIII metals, e.g. manganese or ruthenium and examples of ligand Lg (each Lg may be the same or different) are halogen, especially chlorine or bromine.

Typical formulae for the anion ([M(Lg)_{P}D include ([M(Cl)_{P}D or ([M(Br)ₚp), especially ([M(Cl)₄]^{2″} or ([M(Br)₄]^{2′}). E.g. where the metal is manganese, the anions may, for example, be of formulae ([Mn(Cl)₄]^{2″}) or ([Mn(Br)₄]^{2″}).

Other examples of metals, include lanthanides such as cerium or europium. In these cases the anion ([M(Lg)ₚ]^{m-}) may have the formula ([M(Lg)₆]³⁻). E.g ([M(Lg)ₚf ⁻) may have the formula ([M(Cl)₆]³⁻) or ([M(Br)₆]³—). More specifically in the case of cerium, the anion ([M(Lg)ₚ]^{m-}) could have the formula ([Ce(Cl)₆]³⁻) or ([Ce(Br)₆]³⁻). In the case of europium the anion ([M(Lg)ₚ]^{m-}) could have the formula ([Eu(Cl)₆]³⁻) or ([Eu(Br)₆]³⁻).

Physical properties such as melting point, solubility in organic solvents and light-emitting characteristics of the light-emitting complex salts of the invention are dependent to a large extent on the size, structure and hydrophobicity of the organic cation [Org]ⁿ⁺.

Generally the molecular weight of [Org]ⁿ⁺ should be less than 1000, preferably less than 500 and most preferably less than 250. Thus when [Org]ⁿ⁺ is a tertiary ammonium or tertiary phosphonium cation of formulae (NR⁹R^{h}RⁱR^{j})⁺ or (PR⁹R^{R}ⁿR¹RJ)⁺ as defined below, the groups R^{g} R^{h} Rⁱ and R^{j} will preferably each contain less than 30 carbon atoms, and most preferably less than 20 carbon atoms. In preferred embodiments of complex light emitting salts according to the invention of formulae (NR⁹R^{h}RⁱR^{j})⁺ or (PR⁹R^{h}RⁱR^{j})⁺ one of R⁹, R \ R^{j} and R^{j} will have from 1 to 20 carbon atoms and the remainder from 1 to 6 carbon atoms. In particularly preferred compounds, one of R⁹, R^{h}, Rⁱ and R^{j} will have from 10 to 20 carbon atoms and the remainder from 1 to 6 carbon atoms.

In preferred complex salts according to the invention [Org]ⁿ⁺ is heterocyclic cation, especially ones comprising a heterocyclic nucleus selected from pyridine, pyridazine, pyrimidine, pyrazine, imidazole, pyrazole, oxazole and triazole.

Again the molecular weight of [Org]ⁿ⁺ should be less than 1000, preferably less than 500 and most preferably less than 250. Thus when [Org]^{π+} is a substituted heterocyclic nucleus selected from pyridine, pyridazine, pyrimidine, pyrazine, imidazole, pyrazole, oxazole and triazole the substituents (e.g. substituents R^{a}, R^{b}, R^{c}, R^{d}, R^{e} and R^{f} defined below) will preferably each contain less than 30 carbon atoms, and most preferably less than 20 carbon atoms. In preferred embodiments of complex light emitting salts according to the invention when [Org]ⁿ⁺ is a a substituted heterocyclic nucleus selected from pyridine, pyridazine, pyrimidine, pyrazine, imidazole, pyrazole, oxazole and triazole, one of the substituents (e.g. substituents R^{a}, R^{b}, R^{c}, R^{d}, R^{e} and R^{f} defined below) will have from 1 to 20 carbon atoms and the remainder from 1 to 6 carbon atoms. In particularly preferred compounds, one of R^{a}, R^{b}, R^{c}, R^{d}, R^{e} and R^{f} will have from 10 to 20 carbon atoms and the remainder from 1 to 6 carbon atoms.

The majority of the complex salts of the invention are capable of forming ionic liquids.

The term “ionic liquid” as used herein refers to a liquid that is capable of being produced by melting a solid, and when so produced, consists solely of ions. Ionic liquids may be derived from organic salts, especially salts of heterocyclic nitrogen-containing compounds. Thus, in the context of the present invention, Org preferably comprises a heterocyclic nucleus.

An ionic liquid may be formed from a homogeneous substance comprising one species of cation and one species of anion, or can be composed of more than one species of cation and/or anion. Thus, an ionic liquid may be composed of more than one species of cation and one species of anion. An ionic liquid may further be composed of one species of cation, and one or more species of anion. Thus the mixed salts of the invention can comprise mixed salts containing anions and cations in addition to the specified [OrgJⁿ⁺ cations and [M(Lg)ₚ]^{m″} anions. They may further comprise mixed salts in which more than one species of the specified [Org]ⁿ⁺ cations and [M(Lg)ₚ]^{m′} anions are present.

Thus, in summary, the term “ionic liquid” as used herein may refer to a homogeneous composition consisting of a single salt (one cationic species and one anionic species) or it may refer to a heterogeneous composition containing more than one species of cation and/or more than one species of anion.

The term “ionic liquid” includes compounds having both high melting temperature and compounds having low melting points, e.g. at or below room temperature (i.e. 15-30° C.). The latter are often referred to as “room temperature ionic liquids”.

The complex salts of the invention generally are not preferred to be “room temperature ionic liquids” as normally the light emitting profiles are diminished or are lost when the complex salts are in the liquid state. Surprisingly, a fluorescent complex salt according to the invention has been found to retain its fluorescence even when in the liquid state as will be described below.

As indicated, preferred complex salts according to the invention comprise complex ions of alkylated or polyalkylated heteroaryl compounds, such as alkylated pyridine, pyridazine, pyrimidine, pyrazine, imidazole, pyrazole, oxazole and triazole. Thus, examples of such cations include those having the following formula:

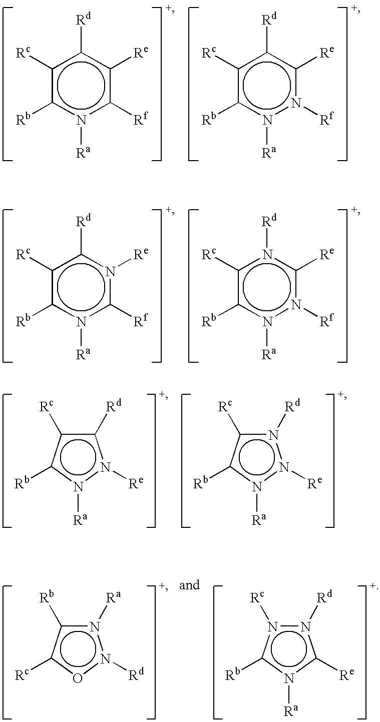

wherein
R^{a} is a Ci to C₄₀, (preferably C₁ to C₂₀ and more preferably C₄ to C12) straight chain or branched alkyl group or a C₃ to C₈ cycloalkyl group, wherein said alkyl or cycloalkyl group which may be substituted by one to three groups selected from: C₁ to C₆ alkoxy, C₆ to C₁₀ aryl, CN, OH, NO₂, C₁ to C30 aralkyl and C₁ to C₃₀ alkaryl;

R^{a}, R^{o}, R^{d}, R^{e} and R^{f} can be the same or different and are each independently selected from
hydrogen,
a C₁ to C₄₀, (preferably C₁ to C₂o and more preferably C₄ to C₁₂) straight chain or branched alkyl group, a C₃ to C₈ cycloalkyl group, or a C₆ to C₁₀ aryl group, wherein said alkyl, cycloalkyl or aryl groups are unsubstituted or may be substituted by one to three groups selected from:
C₁ to C₆ alkoxy, C₆ to C₁₀ aryl, CN, OH, NO₂, C₇ to C₃₀ aralkyl and C₇ to C₃₀ alkaryl, or
any two of R^{b}, R^{c}, R^{d}, R^{e} and R^{f} attached to adjacent carbon atoms form a methylene chain —(CH₂)_{q}— wherein q is from 2 to 8, especially 3, 4 or 5.

Preferably, R^{a} is an unsubstituted alkyl or cycloalkyl group as defined above. R^{b}, R^{c}, R^{d}, R^{e} and R^{f} are preferably hydrogen or C₁₋₁₀ alkyl. Examples of such preferred compounds are ones in which one or two of of R^{b}, R^{c}, R^{d}, R^{e} and R^{f} represent C₁₋₁₀ alkyl and the other three or four of R^{b}, R^{c}, R^{d}, R^{e} and R^{f} represent hydrogen.

In preferred complex salts of the present invention, the cation is 1,3-dialkylimidazolium. Other preferred cations include other substituted pyridinium or alkyl- or poly-alkylpyridinium, alkyl imidazolium, imidazole, alkyl or poly-alkylimidazolium, alkyl or polyalkylpyrazolium, ammonium, alkyl or polyalkyl ammonium, alkyl or poly-alkyl phosphonium cations.

Particularly preferred ionic liquids are imidazolium, pyridinium or pyrazolium salts. Thus those based on imidazolium cations may suitably have the formula: 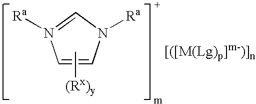
wherein
each R^{a} may be the same or different and each is independently selected from Ci to C₄₀ straight chain or branched alkyl which may be substituted by one to three groups selected from: Ci to C₆ alkoxy, C₆ to c-₁₀ aryl, CN, OH, NO₂, Ci to C₃₀ aralkyl and Ci to C₃₀ alkary
R^{x} represents a Ci to Ci₀ straight chain or branched alkyl which may be substituted by one to three groups selected from: C₁ to c₆ alkoxy, C₆ to C₁₀ aryl, CN, OH, NO₂, Ci to Ci₀ aralkyl and C₁ to C₁₀ alkaryl;
y is 0, 1, 2 or 3;
M, Lg, m, n and p are as previously defined.

Those based on pyrazolium may suitably have the formula:

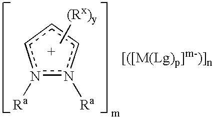

wherein
each R^{a} may be the same or different and each is independently selected from C₁ to C₄₀ straight chain or branched alkyl which may be substituted by one to three groups selected from: c-i to Ce alkoxy, c₆ to C₁₀ aryl, CN, OH, NO₂, C₁ to C₃₀ aralkyl and c-i to C₃o alkary
R^{x} represents a Ci to C₁₀ straight chain or branched alkyl which may be substituted by one to three groups selected from: Ci to C₆ alkoxy, c6 to C₁₀ aryl, CN, OH, NO₂, C₁ to C₁₀ aralkyl and C₁ to C₁₀ alkaryl;
y is o, 1, 2 or 3;
M, Lg, m, n and p are as previously defined.

Also suitable are complex salts based on pyridinium cations having the formula: 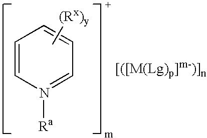
wherein
R^{a} is selected from Ci to C₄o straight chain or branched alkyl which may be substituted by one to three groups selected from: Ci to C₆ alkoxy, C₆ to C₁₀ aryl, CN₁OH, NO₂, Ci to C₃₀ aralkyl and C₁ to C₃₀ alkaryl;
R^{x} represents a Ci to C1₀ straight chain or branched alkyl which may be substituted by one to three groups selected from: C₁ to C₆ alkoxy, C₆ to C-io aryl, CN, OH, NO₂, C₁ to C₁₀ aralkyl and C₁ to C₁₀ alkaryl;
y is 0, 1, 2 or 3;
M, Lg, m, n and p are as previously defined.

Preferably, in the above compounds, R^{a} is independently selected from C₁ to C₄₀, preferably C₁ to C₂₀, and even more preferably, C₄ to C₁₂, straight chain or branched alkyl.

In another exemplary class of compound according to the invention ([Org]ⁿ⁺) may be a quaternary ammonium or phosphonium ion (R⁹RⁿRWN)⁺ or (R⁹R^{h}RⁱR^{j}P)⁺, wherein R^{9′R}^{h}.R¹ and R^{j}, which may be the same or different represent a C₁ to C₄₀, (preferably C₁ to C₂₀ and more preferably C₄ to Ci₂) straight chain or branched alkyl group, a C₃ to C₈ cycloalkyl group, or a C₆ to C₁₀ aryl group, wherein said alkyl, cycloalkyl or aryl groups are unsubstituted or may be substituted by one to three groups selected from: C₁ to C₆ alkoxy, C₆ to C₁Oaryl, CN, OH, NO₂, C₇ to C₃₀ aralkyl and C₇ to C₃₀ alkaryl, or any two of R^{e}, R^{f}, R⁹, R^{h} form a methylene chain —(CH₂)_{q}— wherein q is from 2 to 8, especially 3, 4 or 5.

Preferably, R⁹, R^{h}, R¹ and R^{j} represent substituted or unsubstituted alkyl or cycloalkyl or phenyl groups. The preferred alkyl and cycloalkyl groups preferably contain from 1 to 10 carbon atoms. Examples of preferred compounds are ones in which one, or two or three of R⁹, R^{h}, R¹, and R^{j} represent Cr₁₀ alkyl and the other one, two or three represent C1-₆ alkoxy-substituted C₁₋₁₀ alkyl.

### DESCRIPTION OF FIGURES

The invention will now be described in more detail with particular reference to the accompanying drawings, in which:

FIG. 1 is a photograph of a sample of a manganese(II) bromide room-temperature ionic liquid of formula

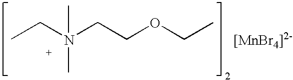

FIG. 2 is a photograph of the sample shown in FIG. 1 alongside a sample of [emim]₂[MnBr₄];

FIG. 3 is a photograph of two tetrabromomanganate salts, and illustrates the difference between compounds which are non-luminescent and luminescent under UV irradiation. The colour is thought to be due to a ⁴T_{Tg}-⁶A_{Tg} Mn 3d transition (⁶A tg is the ground state).

FIG. 4 is a diagram showing the main transition involved in manganese(II) luminescence;

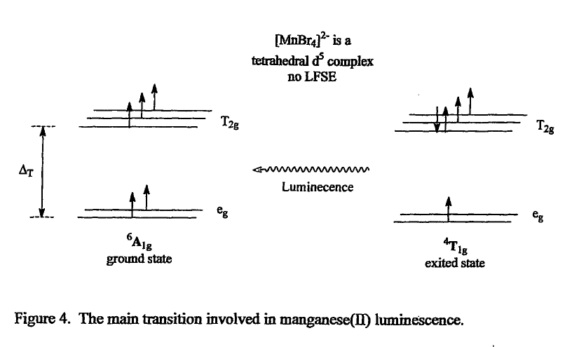

FIG. 5 shows the UV absorbance spectrum of [emim]₂[MnBr₄] and 1-ethyl-2,3-dimethylimidazolium tetrabromomanganate(II), OeUmJm]₂[MnBr₄];

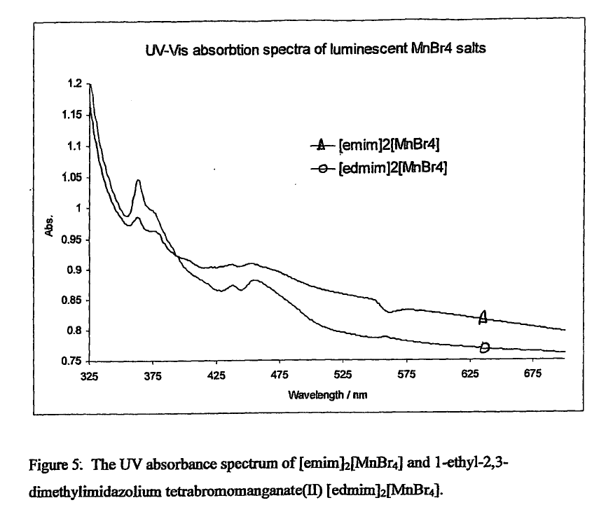

FIG. 6 is a photograph that illustrates the phosphorescence colours of [emim]₂ [MnBr₄], [C₄pyfc [MnBr₄] and [edmim]₂[MnBr₄] (left to right).

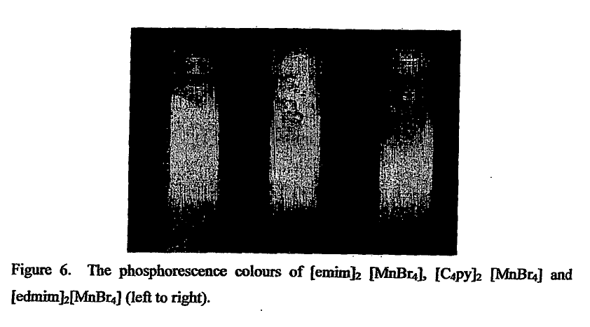

The two compounds in FIG. 5 show phosphorescence (approx 1 millisecond) at 510 and 527 nm as determined on a fluorimeter. The absorptions in the 450 and 370 nm regions are d-d transitions and the strong absorbance at <325 nm is due to Mn—Br charge transfer processes. As illustrated by FIG. 6 the structure of the cation can affect the phosphorescence colour;

FIG. 7 are photographs illustrating the changing crystal structure of [C₁₈DBU]₂[MnBr₄];

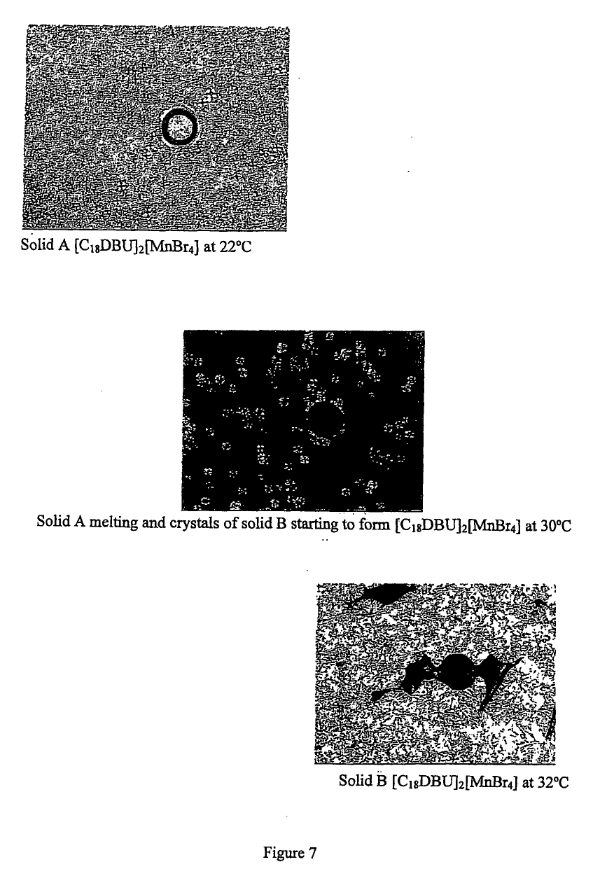

FIG. 8 is a photograph showing the two luminescent complexes of Examples 16 and 19. (Eu-red, Ce-Violet);

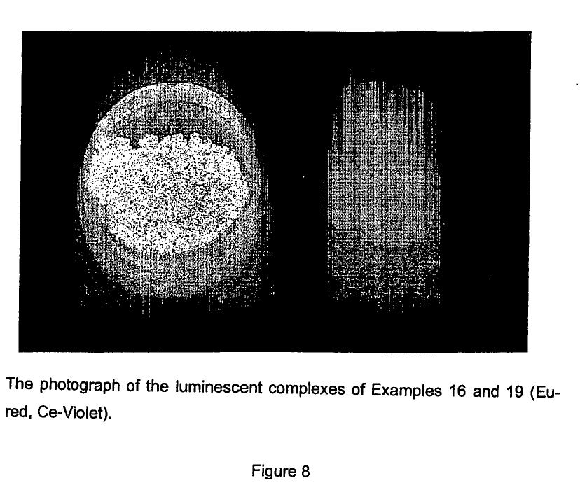

FIG. 9 is a photograph that illustrates the luminescence of [Cn pyridinium]₂[MnBr₄] salts under the uv lamp (n=18, 4, 2 from left to right) and [C₂ lutidinium]₂[MnBr₄] (far right);

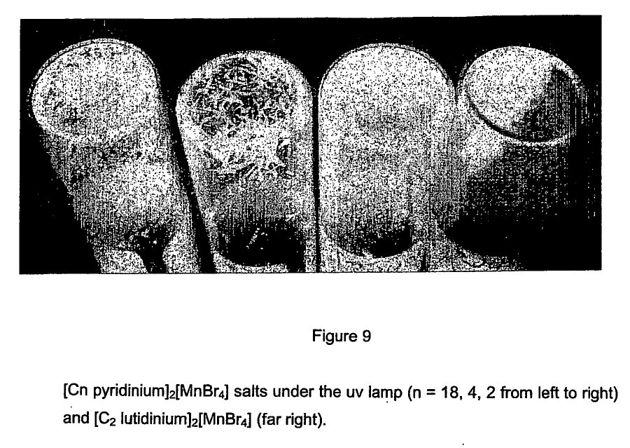

FIG. 10 is a photograph showing [Cn pyridinium]₂[MnBr₄] salts in daylight (n=18, 4, 2 from left to right) and [C₂ lutidinium]₂[MnBr₄] (far right);

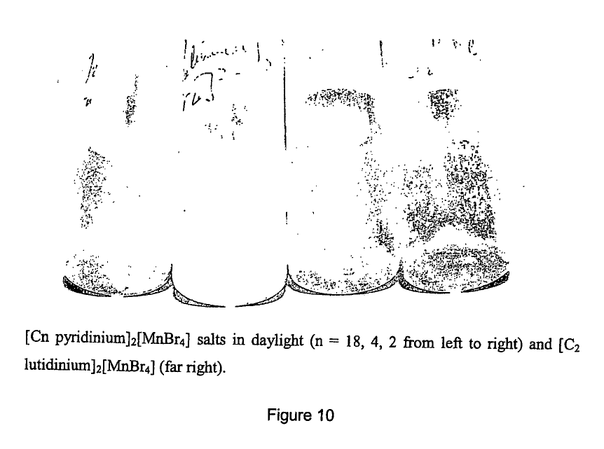

FIG. 11 is a photograph showing the difference in luminous intensity between [emim]₂[MnCl₄] (left) and [emim]₂[MnBr₄] (right);

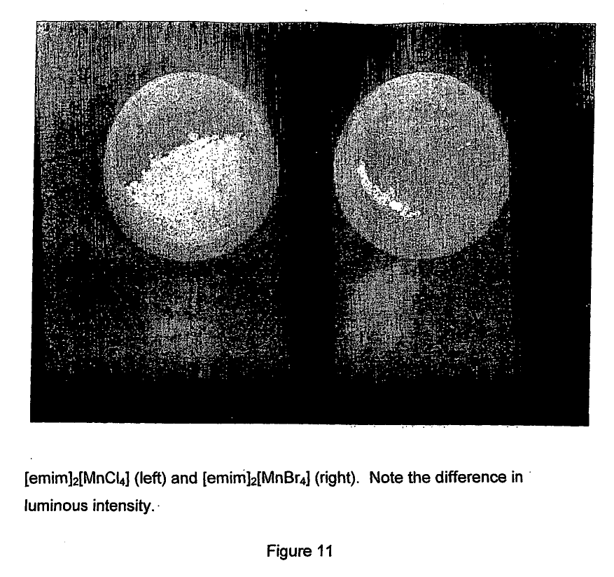

FIG. 12 is a photograph showing [Ci₄mim]₂[MnCl₄] at 130° C. in liquid crystalline phase (possible Smectic A) (top); and [C₁₄mim]₂[MnCl₄] at 64° C. in liquid crystalline phase (possible Smectic A). Rhombic crystals of [Ci₄mim]₂[MnCl₄] growing from liquid crystals phase;

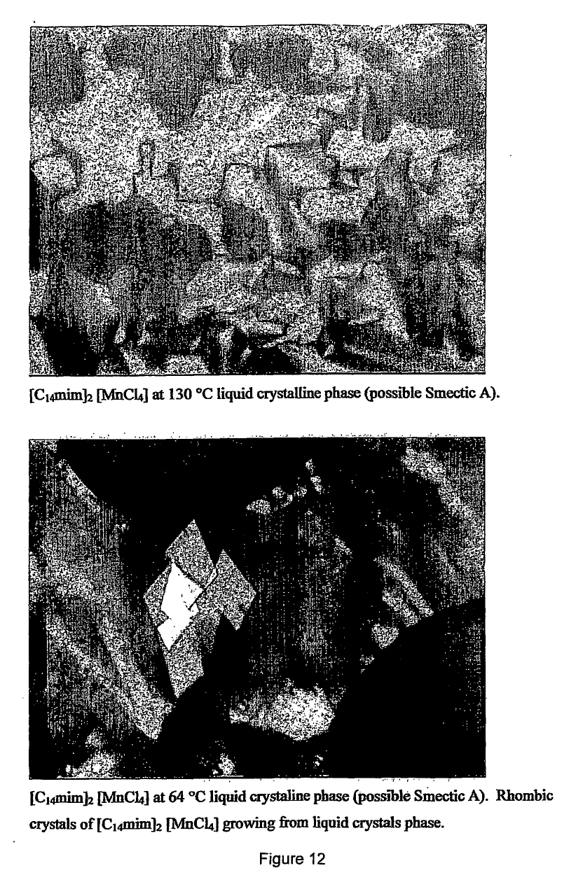

FIG. 13 is a photograph showing [Ci₈mim]₂[MnBr₄] (left) at 100° C. in liquid crystalline phase possible Smectic A) and [Ci₈mim]₂[MnBr₄] solid (right) phase at 74° C. during slow crystallisation from liquid crystal phase;

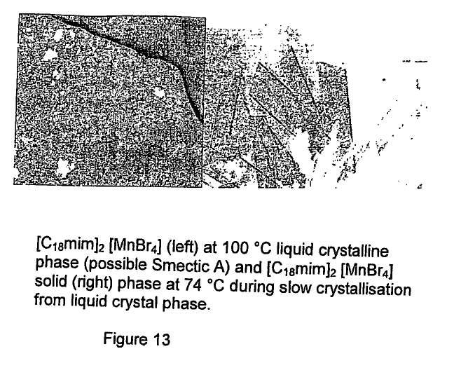

FIG. 14 is a photograph that illustrates the luminous colours of Front [C_{6′}6.6,iOP]₃[CeCl₆], left [C_{6′6′6′}IoP]₃[EuCl₆], right [C_{4′}4,_{4′}I₆P]₂[MnBr₄]; and

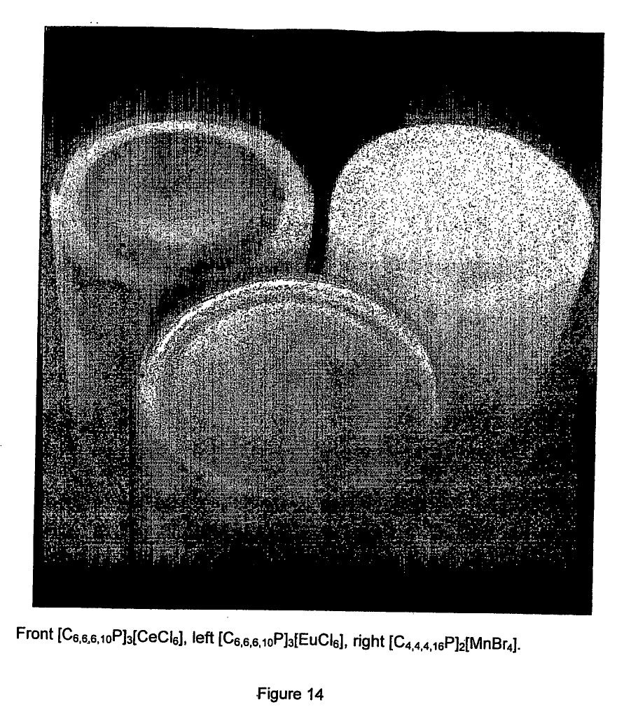

FIG. 15 shows the uv-vis absorption spectrum for [C6,6,_{6′}ioP]₃[CeCl6].

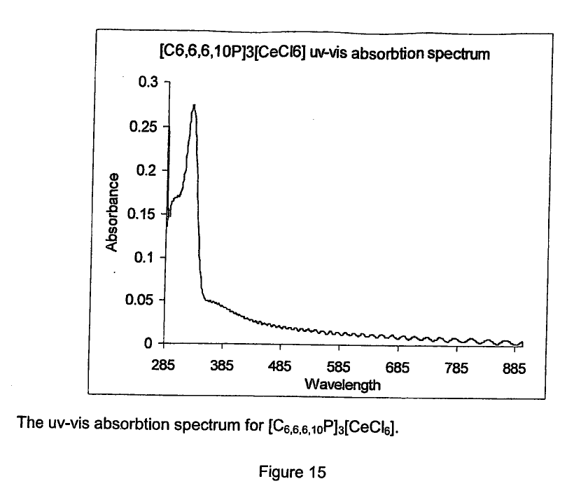

### ANALYSIS TECHNIQUES AND GENERAL SYNTHETIC PROCEDURES

Analysis Techniques

NMR

Manganese(II) is paramagnetic and interferes with the magnetic field in the NMR spectrometer. It is possible to obtain ¹H and ¹³C NMR spectra, but the peaks are extremely broadened and subject to a slight paramagnetic shift.

Elemental Analysis

This technique gives the chemical formula and confirms that in the case of manganese halide-based complex salts of the invention, the most stable complex is a 2:1 [Org]⁺ to [MnX₄]²⁻ complex.

UV Absorbance Spectroscopy

The technique used involved sandwiching the solid between two glass slides (a solvent cannot be used as this quenches the luminescence).

Luminescence Spectrometry

It is possible to obtain both excitation and emission spectra (i.e. absorption and luminescence spectra) by this technique, which provides information about how the cation influences the anions luminescence. ft is also possible to determine if phosphorescence is occurring by measuring the emission after a predefined time delay. Lifetimes in the order of 1 millisecond have been observed for [emim]₂[MnBr₄].

Differential Scanning Calorimetry

This gives the melting points and transition temperatures of the compounds. The luminescence shows significant temperature dependence and it is possible to associate specific transitions with the switching on or off of the luminescence. The technique also gives indirect information on the purity of the complex.

Polarising Microscopy.

Polarising microscopy may be used in the analysis of liquid crystalline luminescent complexes and gives information about purity and transition temperatures.

### GENERAL PREPARATIONS

Manganese complexes

The halide salt of an organic cation (4 mmol) is mixed with the corresponding anhydrous manganese(II) halide salt (2 mmol) in methanol (2.5 cm³). This was stirred while gently heating on a hotplate until all the manganese(II) halide had dissolved. The methanol was boiled off by heating (150° C.) and the crude [organic cation]₂[MnX₄] cooled. The solid tetrahalomanganate(II) salts were recrystallised from boiling ethyl acetate (cations containing long alkyl chains>Ce) or from isopropanol/methanol mixtures (<C₈). The crystalline solids were then heated at 80-120° C. under vacuum (5 mmHg) to remove traces of solvent.

Europium and Cerium Complexes

The halide salt of an organic cation (3 mmol) was mixed with the corresponding anhydrous europium or cerium (II) halide salt (1 mmol) in methanol (10.0 cm³). This was stirred while gently heating on a hotplate until all the lanthanide (III) halide had dissolves. The methanol was boiled off by heating (150° C.) and the crude [organic cation⁺]3[MXe]^{3″} cooled. The solid hexahaloeuropium or cerium (III) salts were recrystallised from boiling ethyl acetate (cations containing long alkyl chains>C₈) or from isopropanol/methanol mixtures (<Cs). The crystalline solids were then heated at 80-120° C. under vacuum (5 mmHg) to remove traces of solvent.

Manganese Halide Complex Salts—Bulk Appearance

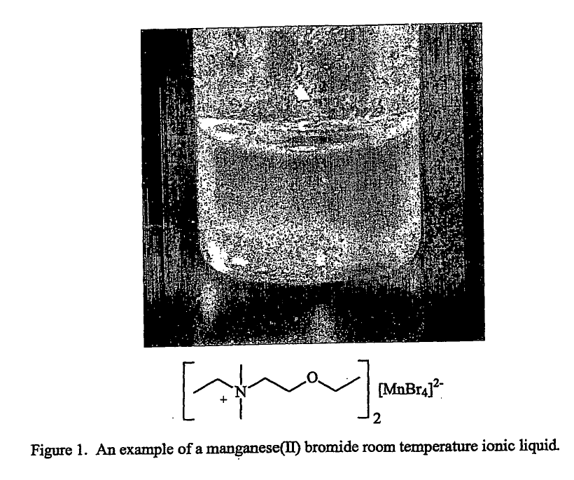

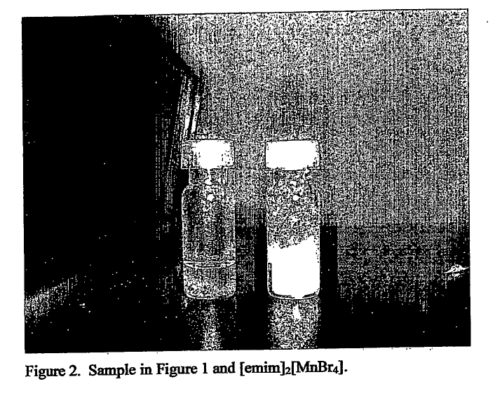

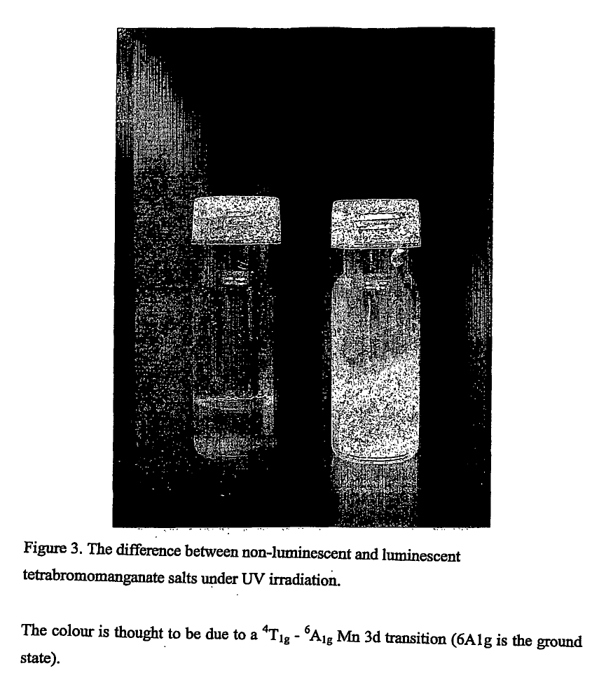

A number of tetrabromomanganese(II) and tetrachloromanganese(II) salts were made by mixing a 2:1 molar ratio of an organic bromide salt with manganese(II) bromide, or an organic chloride salt with manganese(II) chloride, respectively, and heating. Some of the compounds were found to be strongly luminescent in the solid phase. In general, the bromides were considerably more luminescent than the chlorides. An example of a room temperature manganese(II) ionic liquid is given in FIG. 1. The yellow/brown colour is due to a weak d-d absorption transition in the blue part of the spectrum. FIG. 2 shows the difference in colour between the non-luminescent sample in FIG. 1 and the luminescent [emim]2[MnBr₄] in daylight. As can be seen, the luminescence makes the sample appear bright yellow. FIG. 3 shows the colours under long wave UV irradiation. As can be seen, the [emim]₂[MnBr₄] is intensely luminescent in the green part of the visible spectrum.

Sulfonium manganese (II) halide slats were prepared as above with the exception of reactions with a disulfinyl compound where the molar ration was 1:1.

Physical Properties of Individual Manganese(II) Halide Complexes

A range of manganese complexes have been made and their properties are described and listed individually. A similar synthesis technique was used for all of the manganese chloride and manganese bromide salts.

The following specific examples illustrate the invention.

### EXAMPLES

Using the procedures described in General Procedures above, the following complexes were prepared:

### Example 1

### [EmJm]₂[MnBr₄]

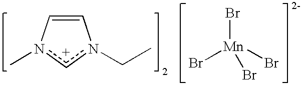 [0107] Appearance: Yellow/green crystalline solid in daylight which changes to yellow/brown above 65° C.
Elemental Analysis: C, 24.03%; H, 3.66%; N, 9.48%. (Theoretical C, 24.15%; H, 3.72%; N, 9.39%).
DSC: mp=163.6° C. (4.7 Jg⁻¹); solid-solid transitions 117.4° C. (0.2 Jg⁻¹) and 64.7° C. (34.3 Jg⁻¹).
Luminescence: Intense green phosphorescence. λmax=510 nm emission; 363, 376 and 455 nm excitation (same as UV absorption spectrum).

### Example 2

### [EcJmIm]₂[MnBr₄]

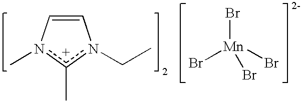 [0112] Appearance: Yellow/green crystalline solid in daylight which changes to yellow brown above 117° C.
Elemental Analysis: C, 27.09%; H, 4.18%; N, 9.25%. (Theoretical C, 26.91%; H, 4.19%; N, 8.97%).
DSC: mp=189.8° C (3.4 Jg⁻¹); solid-solid transition 116.5° C. (35.0 Jg⁻¹). There is also another form with a transition at 87.0° C. which forms slowly.
Luminescence: Intense yellow-green phosphorescence, λₘₐₓ=527 nm emission; 363, 376 and 456 nm excitation (same as UV absorption spectrum).

### Example 3

### [Emim]₂[MnCl₄]

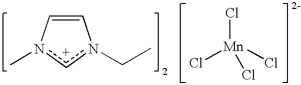 [0117] Appearance: Off-white crystalline solid.
Elemental Analysis:
DSC: mp=129.8° C (2.8 Jg⁻¹); solid-solid transitions 78.8° C. (49.2 Jg-¹) or 48.0° C (46.7 Jg⁻¹). Only one of these solid-solid transitions occurs on heating, depending on the crystalline polymorph formed on freezing.
Luminescence: Moderate blue-green luminescence, λₘₐₓ=528 and 416 (weak) nm emission; 328, 360, 450 and 482 nm excitation. Luminescence disappears above solid-solid transition temperature.

### Example 4

### [C₃ITiJm]₂[MnBr₄]

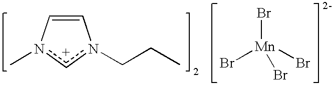 [0122] Appearance: Yellow/green crystalline solid in daylight, below melting point. Melts to pale yellow/brown oil.
Elemental Analysis:
DSC: mp=49.6° C. (33.5 Jg⁻¹).
Luminescence: Intense green luminescence which disappears on melting.

### Example 5

### [C₄mim]₂[MnBr₄]

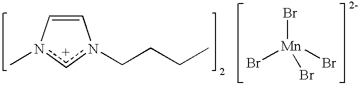 [0127] Appearance: Pale yellow/brown oil at room temperature. Remains as an ionic liquid down to −20° C.
Elemental Analysis:
DSC: mp<−20° C.
Luminescence: No luminescence.

### Example 6

### [Ci₂InJm]₂[MnBr₄]

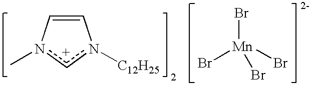 [0132] Appearance: Pale yellow/brown mushy solid.
Elemental Analysis:
DSC:
Luminescence: Weak green luminescence.

### Example 7

### [C₁₄IHilTi]₂[IVInCl₄]

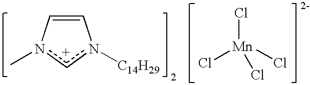 [0137] Appearance: Off-white waxy solid.
Elemental Analysis:
DSC: mp=62.2° C. (93 Jg⁻¹). No evidence of liquid crystal phase.
Luminescence: Weak green luminescence.

### Example 8

### [Ci₆mim]2[MnCl₄]

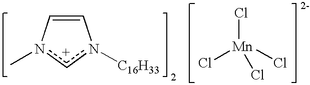 [0142] Appearance: Off-white waxy solid.
Elemental Analysis:
DSC: mp=71.2° C (99 Jg⁻¹). No evidence of liquid crystal phase.
Luminescence: Weak green luminescence.

### Example 9

### [Ci₈mim]₂[MnCl₄]

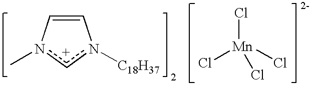 [0147] Appearance: Off-white waxy solid.
Elemental Analysis:
DSC:
Luminescence: Weak green luminescence.

### Example 10

### [C₂PyHdJmUm]₂[MnBr₄]

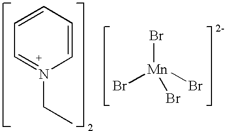 [0152] Appearance: Yellow/green crystalline solid in daylight which changes to yellow/brown above 108° C.
Elemental Analysis:
DSC: mp=155.8° C. (1.2 Jg⁻¹); solid-solid transitions 131.0° C. (2.7 Jg-¹) and 107.7° C. (47.9 Jg⁻¹).
Luminescence: Intense green phosphorescence. Above 108° C., no luminescence observed. λmₐχ=512 nm emission; 363, 375 and 456 nm excitation.

### Example 11

### [C₂IUtIdJmUm]₂[MnBr₄]

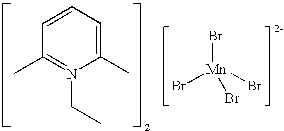 [0157] Appearance: Bright yellow crystalline solid in daylight which changes to yellow/brown above 108° C.
Elemental Analysis:
DSC: mp=193.0° C. (6.1 Jg⁻¹); solid-solid transitions 181.4° C. (25.7 Jg-¹) and 166.4° C. (6.4 Jg⁻¹).
Luminescence: Intense yellow-green luminescence.

### Example 12

### [C₄pyridinium]₂[MnBr₄]

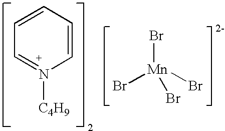 [0162] Appearance: Bright yellow crystalline solid in daylight which changes to pale yellow above 108° C.
Elemental Analysis:
DSC: mp=100.2° C (53.9 Jg⁻¹)
Luminescence Intense green luminescence up to 100° C.

### Example 13

### [C₂pyrazolium]₂[MnBr₄]

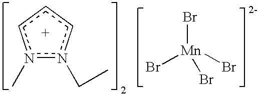 [0167] Appearance: Yellow crystalline solid in daylight which changes to yellow/brown above 108° C.
Elemental Analysis: C, 24.26%; H, 3.64%; N, 9.57%. (Theoretical C, 24.15%; H, 3.72%; N, 9.39%).
DSC: mp=195.5° C. (9.4 Jg⁻¹); solid-solid tr. 86.9° C (2.9 Jg-¹) and 44.5° C. (13.2 Jg⁻¹). Decomposes above 205° C.
Luminescence: Intense green phosphorescence. Above 108° C., no luminescence observed, λₘₐₓ=512 nm emission; 363, 375 and 456 nm excitation.

### Example 14

### [C₄DBU]₂[MnBr₄]

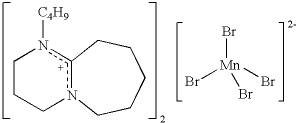 [0172] Appearance: Yellow/green solid.
Elemental Analysis:
DSC: mp=54.5° C. (30.5 Jg⁻¹).
Luminescence: Intense green luminescence in solid phase

### Example 15

### [C₁₈DBU]₂[MnBr₄]

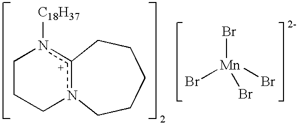 [0177] Appearance: White waxy powder.
Elemental Analysis:
DSC: mp=79.1° C. (42.9 Jg⁻¹). On freezing, i crystallises to the Solid A phase (below 30° C). On heating, Solid A melts at 35.2° C. (40.6 Jg⁻¹) and immediately re-freezes (−41.5 Jg⁻¹) to solid B. On prolonged standing, the first temperature ramp on the DSC appears to show the existence of other polymorphs.
Luminescence: Moderate green luminescence in both solid phases

### Example 16

### [C₆mJm]₃[CeCl₆]

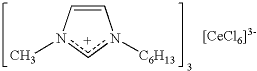 [0182] Appearance: white crystalline solid.
DSC: mp=165-170° C. and decompose above 300° C.
Luminescence: weak violet luminescence in solid phase.

### Example 17

### [Bu₄N]₃[CeCl₆]

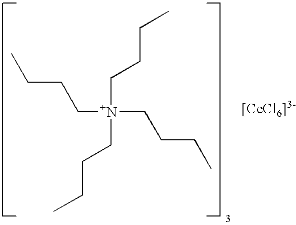 [0186] Appearance: white crystalline solid.
DSC: mp=271° C. and decomposes above 350° C.
Luminescence: strong blue luminescence in solid phase. Absorbs water vapour from the air to form a hydrate which has a weaker violet luminescence.

### Example 18

### [Ce.e.e.ioPMCeCle]

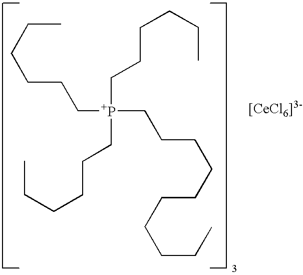 [0190] Appearance: Pale yellow room temperature ionic liquid.
DSC: not yet determined, mp=<20° C.
Luminescence: strong blue luminescence in liquid phase. Absorbs water vapour from the air to form a hydrate which has a weaker violet luminescence. Excitation maxima at 311 and 350 mn, emission maxima at 502 nm. This emission peak is red shifted slightly due to overlap of the excitation and emission spectra.
   The type of luminescence was determined to be either a very short lived phosphorescence with a half life of 10 microseconds or fluorescence.

### Example 19

### [C₆mim]₃[EuCl₆]

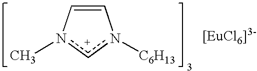 [0195] Appearance: white crystalline solid.
DSC: mp=169.5×(26 jg⁻¹) and decompose above 300° C.
Luminescence: weak red luminescence in solid phase.

### Example 20

### [C_{6′6′6′I}oP]₃[EuCI₆]

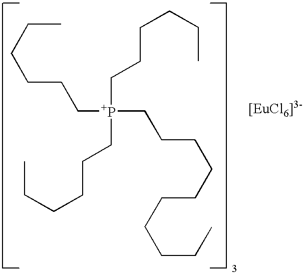 [0199] Appearance: Colourless room temperatue ionic liquid.
DSC: not yet determined, mp=<20° C.
Luminescence: Red luminescence in liquid phase. Absorbs water vapour from the air to form a hydrate. This still shows some luminescence. Excitation maxima at 530, 460 and 400 nm, emission maxima at 590, 610, 650 and 700 nm.
   The type of luminescence was determined to be phosphorescence with a half life of 1.77 microseconds.

Compounds of the invention may be used in a wide range of industrial applications that make use of their light-emitting characteristics. Examples include imaging and display devices, electro-optical devices and assay procedures. Thus, the fluorescent, phosphorescent and electroluminescent compounds may be used in the manufacture of cathode ray tubes, fluorescent tubes, X-ray-imaging screens, radiation detectors, toys and other recreational devices, signs, light-emitting solid state devices etc. Specific examples include the displays of mobile telephones, calculators, computer screens and flat-screen television displays

More specific applications include organic light emitting diodes (OLEDS) in which the complex salts of the invention can be incorporated as discrete layers or dopants. Other uses include:
biological markers and reagents (e.g. to form tagged reagents);
luminescent devices useful in hobbies, e.g. fishing lures;
for use in detectors for explosives (e.g. TNT) or radiation;
safety devices;
as additives for plastics, inks and paints;
security devices;
as coatings for ophthalmic lenses.

1. A complex salt having the formula
([Org]ⁿ⁺)ₘ·([M(Lg)ₚ]^{m-})ₙ
wherein m=1, 2, 3 or 4;
n=1 or 2;
p=3, 4, 5 or 6;
M is a metal;
each Lg, which may be the same or different, represents a ligand; and
[Org]ⁿ⁺ represents an organic cation,
and wherein said complex salt (1) exhibits at least one light emitting property selected from (a) fluorescence, (b) phosphorescence, and (c) electroluminescence when in the solid state, (2) has a melting point below 250° C. and (3) are capable of forming ionic liquids when molten.

2. A complex salt according to claim 1 having a melting point below 200° C.

3. A complex salt according to claim 1 having a melting point below 180° C.

4. A complex salt according to claim 1 having a melting point below 150° C.

5. A complex salt according to claim 1 having a melting point below 125° C.

6. A complex salt according to claim 1 having a melting point below 100° C.

7. A complex according to claim 1 wherein m is 2.

8. A complex according to claim 1 wherein m is 1.

9. A complex according to claim 1 wherein n is 1.

10. A complex according to claim 1 wherein p is 4, 5 or 6.

11. A complex according to claim 1 wherein p is 4.

12. A complex salt according to claim 1 wherein M is a Group VII or VIII metal.

13. A complex salt according to claim 1 wherein M is manganese or ruthenium.

14. A complex salt according to claim 1 wherein each Lg is halogen.

15. A complex salt according to claim 14 wherein each Lg is Cl or Br.

16. A complex salt according to claim 15 wherein the anion ([M(Lg)ₚ]^{m-}) has the formula ([M(Cl)ₚ]^{m-}) or ([M(Br)ₚ]^{m-}).

17. A complex salt according to claim 16 wherein the anion ([M(Lg)ₚ]^{m-}) has the formula ([M(Cl)₄]²⁻) or ([M(Br)₄]²⁻).

18. A complex salt according to claim 17 wherein the anion ([M(Lg)ₚ]^{m-}) has the formula ([Mn(Cl)₄]²⁻) or ([Mn(Br)₄]²⁻).

19. A complex salt according to claim 1 wherein M is a lanthanide.

20. A complex salt according to claim 19 wherein M is cerium or europium.

21. A complex salt according to claim 19 wherein the anion ([M(Lg)ₚ]^{m-}) has the formula ([M(Lg)₆]³⁻).

22. A complex salt according to claim 21 wherein the anion ([M(Lg)ₚ]^{m-}) has the formula ([M(Cl)₆]³⁻) or ([M(Br)₆]³⁻).

23. A complex salt according to claim 22 wherein the anion ([M(Lg)ₚ]^{m-}) has the formula ([Ce(Cl)₆]³⁻) or ([Ce(Br)₆]³⁻).

24. A complex salt according to claim 22 wherein the anion ([M(Lg)ₚ]^{m-}) has the formula ([Eu(Cl)₆]³⁻) or ([Eu(Br)₆]³⁻).

25. A complex salt according to claim 1 in which [Org]ⁿ⁺ is heterocyclic.

26. A complex salt according to claim 25, wherein [Org]ⁿ⁺ comprises a heterocyclic nucleus selected from pyridine, pyridazine, pyrimidine, pyrazine, imidazole, pyrazole, oxazole, and triazole.

27. A complex salt according to claim 25 wherein [Org]ⁿ⁺ has a structure selected from the following formula: 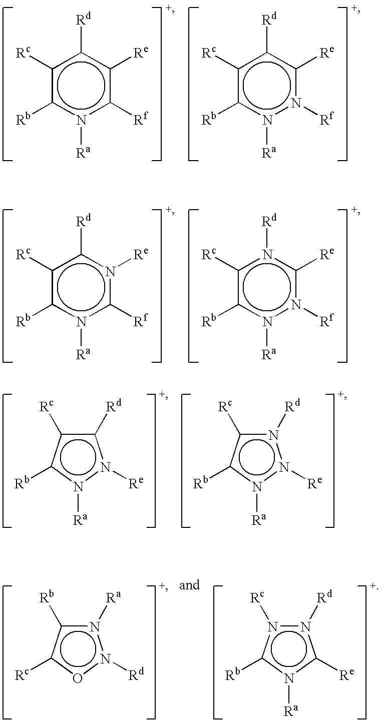
wherein
R^{a} is a C₁ to C₄₀, straight chain or branched alkyl group or a C₃ to C₈ cycloalkyl group, wherein said alkyl or cycloalkyl group which may be substituted by one to three groups selected from: C₁ to C₆ alkoxy, C₆ to C₁₀ aryl, CN, OH, NO₂, C₁ to C₃₀ aralkyl and C₁ to C₃₀ alkaryl;
R^{b}, R^{c}, R^{d}, R^{e} and R^{f} can be the same or different and are each independently selected from hydrogen, a C₁ to C₄₀, straight chain or branched alkyl group, a C₃ to C₈ cycloalkyl group, or a C₆ to C₁₀ aryl group, wherein said alkyl, cycloalkyl or aryl groups are unsubstituted or may be substituted by one to three groups selected from: C₁ to C₆ alkoxy, C₆ to C₁₀ aryl, CN, OH, NO₂, C₇ to C₃₀ aralkyl and C₇ to C₃₀ alkaryl, or
any two of R^{b}, R^{c}, R^{d}, R^{e} and R^{f} attached to adjacent carbon atoms form a methylene chain —(CH₂)_{q}— wherein q is from 8 to 20.

28. A complex salt according to claim 25 having the formula: 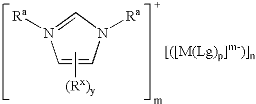
wherein
each R^{a} may be the same or different and each is independently selected from C₁ to C₄₀ straight chain or branched alkyl which may be substituted by one to three groups selected from: C₁ to C₆ alkoxy, C₆ to C₁₀ aryl, CN, OH, NO₂, Cl to C₃₀ aralkyl and C₁ to C₃₀ alkaryl;
R^{x} represents a C₁ to C₁₀ straight chain or branched alkyl which may be substituted by one to three groups selected from: C₁ to C₆ alkoxy, C₆ to C₁₀ aryl, CN, OH, NO₂, C₁ to C₁₀ aralkyl and C₁ to C₁₀ alkaryl;
y is 0, 1, 2 or 3;
m=1, 2, 3 or 4; and n=1 or 2.

29. A complex salt according to claim 25 having the formula: 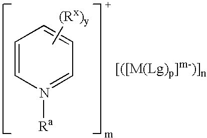
wherein
R^{a} is selected from C₁ to C₄₀ straight chain or branched alkyl which may be substituted by one to three groups selected from: C₁ to C₆ alkoxy, C₆ to C₁₀ aryl, CN, OH, NO₂, C₁ to C₃₀ aralkyl and C₁ to C₃₀ alkaryl;
R^{x} represents a C₁ to C₁₀ straight chain or branched alkyl which may be substituted by one to three groups selected from: C₁ to C₆ alkoxy, C₆ to C₁₀ aryl, CN, OH, NO₂, C₁ to C₁₀ aralkyl and C₁ to C₁₀ alkaryl;
y is 0, 1, 2 or 3;
m=1, 2, 3 or 4; and n=1 or 2.

30. A complex salt according to claim 1 in which [Org]ⁿ⁺ is a phosphonium cation (R^{g}R^{h}RⁱR^{j}P)⁺, wherein R^{g}, R^{h}, Rⁱ and R^{j} can be the same or different and are each independently selected from a C₁ to C₄₀, straight chain or branched alkyl group, a C₃ to C₈ cycloalkyl group, or a C₆ to C₁₀ aryl group, wherein said alkyl, cycloalkyl or aryl groups are unsubstituted or may be substituted by one to three groups selected from: C₁ to C₆ alkoxy, C₆ to C₁₀ aryl, CN, OH, NO₂, C₇ to C₃₀ aralkyl and C₇ to C₃₀ alkaryl

31. A complex salt according to claim 1 in which [Org]ⁿ⁺ is a quaternary ammonium cation (R^{g}R^{h}RⁱR^{j}N)⁺, wherein R^{g}, R^{h}, Rⁱ R^{j} can be the same or different and are each independently selected from a C₁ to C₄₀ straight chain or branched alkyl group, a C₃ to C₈ cycloalkyl group, or a C₆ to C₁₀ aryl group, wherein said alkyl, cycloalkyl or aryl groups are unsubstituted or may be substituted by one to three groups selected from: C₁ to C₆ alkoxy, C₆ to C₁₀ aryl, CN, OH, NO₂, C₇ to C₃₀ aralkyl and C₇ to C₃₀ alkaryl.

32. A complex salt according to claim 12 wherein [Org]ⁿ⁺ is other than tetramethylammonium, tetraethylammonium, tetrabutylammonium, trimethylphenylphosphonium and/or triphenylmethylphosphonium.

33. A complex salt having the formula
([Org]ⁿ⁺)ₘ·([M(Lg)ₚ]^{m-})ₙ
wherein m=1, 2, 3 or 4;
n=1 or 2;
p=3, 4, 5 or 6;
M is a metal;
each Lg, which may be the same or different, represents a ligand; and
[Org]ⁿ⁺ represents an organic cation
with the proviso that when M is Mn, the organic cation [Org]ⁿ⁺ is other than tetramethylammonium, tetraethylammonium, tetrabutylammonium, trimethyl-phenylphosphonium and triphethylmethylphosphonium.

34. A complex salt according to claim 33 wherein M is a lanthanide.

35. A complex salt according to claim 34 wherein M is cerium or europium.

36. A complex salt according to claim 35 wherein [Org[ⁿ⁺ is other than 1-butyl-3-methyl-imidazolium, acetonitrile and/or aluminium chloride-1-methyl-3-ethylimidazolium.

37. A complex salt according to claim 33 wherein M is a Group VII or Group VIII metal.

38. A complex salt according to claim 33 wherein [Org]ⁿ⁺ is other than 1-methyl-3 ethylimidazolium and/or pyridinium.

39. A complex salt according to claim 37 wherein M is ruthenium.

40. A complex salt according to claim 37 wherein M is manganese.

41. A complex salt according to claim 39 wherein [Org]ⁿ⁺ is other than 1-methyl-3-ethylimidazolium.

42. A complex salt according to claim 40 wherein [Org]ⁿ⁺ is other than 1-methyl-3-ethylimidazolium.

43. (canceled)

44. A luminescent display device comprising a light-emitting element comprising a complex salt of claim 1.

45. A set of phosphors, comprising a plurality of different phosphors, each phosphorescing at a different wavelength and each having the formula:
([Org]ⁿ⁺)ₘ·([M(Lg)ₚ]^{m-})ₙ
wherein m=1, 2, 3 or 4;
n=1 or 2;
p=3, 4, 5 or 6;
M is a metal:
each Lg, which may be the same or different, represents a ligand; and
[Org]ⁿ⁺ represents an organic cation,
and wherein said complex salt (1) exhibits at least one light emitting property selected from (a) fluorescence, (b) phosphorescence, and (c) electroluminescence when in the solid state, (2) has a melting point below 250° C. and (3) are capable of forming ionic liquids when molten.

46. A set of 3 phosphors according to claim 45 wherein one compound phosphoresces at a wavelength corresponding to a blue colour, a second at a wavelength corresponding to a red colour, and a third at a wavelength corresponding to a green colour.

47. A complex salt according to claim 19 wherein [Org]ⁿ⁺ is other than tetramethylammonium, tetraethylammonium, tetrabutylammonium, trimethyl-phenylphosphonium and triphethylmethylphosphonium.
